(19) United States
(12) Patent Application Publication
(10) Pub. No.: US 2024/0298915 A1
(43) Pub. Date: Sep. 12, 2024

(54) HIGH FREQUENCY DEVICE FOR 3D MAPPING OF TRACHEA AND LUNGS

(71) Applicant: Ryan Redford, Irvine, CA (US)

(72) Inventor: Ryan Redford, Irvine, CA (US)

(21) Appl. No.: 18/601,813

(22) Filed: Mar. 11, 2024

Related U.S. Application Data
(60) Provisional application No. 63/451,472, filed on Mar. 10, 2023.

(51) Int. Cl. A61B 5/0507; A61B 5/00

(52) U.S. Cl. CPC A61B 5/0507; A61B 5/742; A61B 2503/40

(57) ABSTRACT

Disclosed is a device for non-invasive 3D mapping of target internal body structures using high frequency waves. The device comprises a probe configured for emitting high frequency waves, a receiver for receiving the reflected waves, and a processing unit for calculating the distance between the probe and receiver based on the time delay of the reflected waves. The device also comprises a display for presenting a 3D map of the target internal body structures based on the calculated distances. The device can be used to diagnose various health conditions and guide medical procedures.

### CROSS REFERENCE TO RELATED APPLICATIONS

This application claims priority from U.S. Provisional Application Ser. No. 63/451,472, filed Mar. 10, 2023, the contents of which are incorporated herein in their entirety.

### FIELD OF THE INVENTION

The present disclosure relates to the field of medical imaging. The disclosure has particular utility in connection with a device and method for 3D mapping of target internal body structures such as the trachea and lungs of a human or animal using high frequency waves, and will be described in connection with such utility, although other utilities are contemplated.

### BACKGROUND AND SUMMARY

Tracheobronchial diseases, such as asthma, chronic obstructive pulmonary disease (COPD), and lung and throat cancer are among the leading causes of morbidity and mortality worldwide. Accurate diagnosis and treatment of these diseases requires precise visualization and analysis of the trachea and lungs.

However, such respiratory conditions can be difficult to diagnose and treat due to the internal location of the trachea and lungs. Bronchoscopies, which involve inserting a scope into the airways through the mouth or nose, are commonly used to diagnose and treat respiratory conditions, but they can be uncomfortable for the patient and carry a risk of complications.

Conventional imaging modalities, such as computed tomography (CT) and magnetic resonance imaging (MRI), provide detailed images of the respiratory system, but are often associated with high costs, radiation exposure, and limited accessibility. In addition, these modalities do not provide real-time images and may not capture the dynamic nature of respiratory processes.

High frequency ultrasound has been used in medical imaging for decades and has several advantages, including high resolution, good tissue contrast, real-time imaging, and no radiation exposure. However, current ultrasound systems have limited penetration depth and resolution in the deep tissues of the chest, which makes it challenging to accurately visualize and map the trachea and lungs.

The present disclosure provides a non-invasive alternative for 3D mapping of target internal body structures of a human or animal such as the trachea and lungs. The device comprises a probe configured for emitting high frequency waves, a receiver for receiving the reflected waves, and a processing unit for calculating the distance between the probe and receiver based on the time delay of the reflected waves, and a display for presenting a 3D map of the target structures based on the calculated distances. The high frequency reflected waves can be in the range of 100 MHz to 10 GHz, and the frequency and intensity of the waves can be adjusted using a user interface.

In a further aspect of the present disclosure, the target body structures comprise the trachea or lungs.

In yet a further aspect of the present disclosure, further provides a positioning system for aligning the device with the trachea and lungs.

In yet another aspect of the present disclosure, the target body structures are selected from the group consisting of the heart, liver, pancreas, kidneys, bladder, stomach, intestines, brain and arteries.

In yet another aspect, the target body structures comprise the throat or thyroid.

The device also comprises a display for presenting a 3D map of target interval body structures such as the trachea and lungs based on the calculated distances. The 3D map can be used to diagnose respiratory conditions and guide medical procedures such as bronchoscopies. The device may also comprise a positioning system for aligning the device with the trachea and lungs.

More particularly, the present disclosure provides a high frequency device and method for 3D mapping of target internal body structures of a human or animal such as the trachea and lungs, using high frequency waves in the megahertz range. The device includes a probe that is placed on the surface of the throat, chest or in contact with the skin of the human or animal and emits high frequency waves that penetrate the body structure, i.e., throat or chest wall and propagate through the trachea and lungs. The reflected waves are detected by the probe and used to construct a 3D map of the respiratory system.

The device also may include a user interface that displays the 3D map in real-time, allowing the user to visualize and analyze internal body structures such as the trachea and lungs from various angles and perspectives. The device also may include a database that stores the 3D maps for later analysis and comparison.

More particularly, the present disclosure provides a device for non-invasive 3D mapping of target internal body structures such as the trachea and lungs, comprising: a probe configured for emitting high frequency waves; a receiver for receiving the reflected waves; a processing unit for calculating the distance between the probe and receiver based on the time delay of the reflected waves; and a display for presenting a 3D map of the trachea and lungs based on the calculated distances.

In one aspect of the disclosure the high frequency waves are in the range of 100 MHz to 10 GHz.

In another aspect of the disclosure the device further comprises a user interface for adjusting the frequency and intensity of the high frequency waves.

In still yet another aspect of the disclosure, the device further comprises a positioning system for aligning the device with the target internal body structures.

The present disclosure also provides a method for non-invasive 3D mapping of target internal body structures such as the trachea and lungs using a device including a probe, a receiver, a processing unit and a display as above described, comprising the steps of: emitting high frequency waves from the probe; receiving the reflected waves with the receiver; calculating the distance between the probe and receiver based on the time delay of the reflected waves; and presenting a 3D map of the trachea and lungs on the display based on the calculated distances.

The disclosure also provides a system for diagnosing respiratory conditions and guiding medical procedures, comprising: a device as above described, and including a device for non-invasive 3D mapping of target internal body structures such as the trachea and lungs, comprising: a probe for emitting high frequency waves; a receiver for receiving the reflected waves; a processing unit for calculating the distance between the probe and receiver based on the time delay of the reflected waves; and a display for presenting a 3D map of the target internal body structures based on the calculated distances, and a computer system for storing and analyzing the 3D map of the trachea and lungs.

The device may be used in various settings, including hospitals, clinics, and patient homes, and may be portable and easy to use.

### BRIEF DESCRIPTION OF THE DRAWINGS

Features and advantages of the instant disclosure will be seen from the following detailed description taken in conjunction with the accompanying drawings, wherein:

FIG. 1 is a schematic illustration of a non-invasive 3D mapping device according to an embodiment of the invention; and

FIG. 2 is a flowchart illustrating the method for 3D mapping of a target internal body structure using the non-invasive 3D mapping device according to an embodiment of the invention.

### DETAILED DESCRIPTION OF THE INVENTION

As used herein the terms “transmitter” and “probe” are used interchangeably.

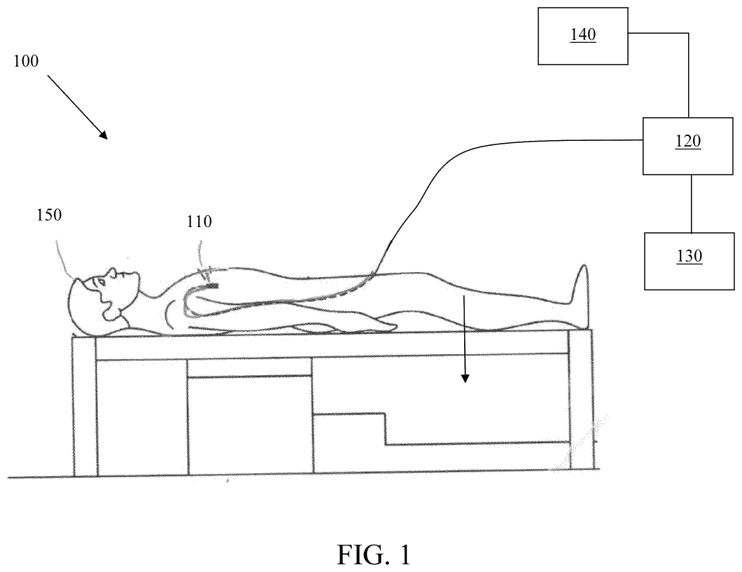

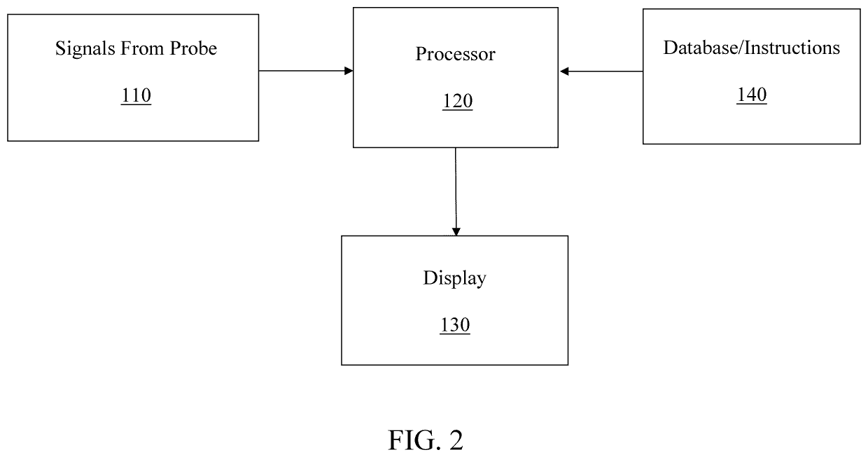

Referring to FIGS. 1 and 2, the non-invasive 3D mapping device 100 according to an embodiment of the invention includes a high-frequency probe 110, a processor 120, a user interface/display 130, and a database 140.

Probe 110 is placed on the skin of a patient 150 over the target internal body structure. For example, probe 110 may be placed on the surface of the chest of a patient 150 over the patient's lungs. Probe 110 includes an array of transducers configured to emit high frequency waves in the megahertz range, such as 1-10 MHz. The high frequency waves penetrate the chest wall and propagate through the lungs. Probe 110 also includes an array of sensors configured to detect reflected waves and transmit the data to the processor 120.

Processor 120 receives the data from the probe 110 and working with data base 140 uses the data to construct a 3D map of the respiratory system and transmits the 3D map to a user interface. In the case where the target internal body structure is the trachea and lungs, the 3D map may include the bronchi, bronchioles, and alveoli, and may show the shape, size, and location of these structures such as a display 130. The processor 120 may also analyze the data to extract various parameters, such as the airway diameter, wall thickness, and tissue stiffness.

The 3D map of the human anatomy generated by the system is based on the principles of wave propagation through different media. When high frequency signals are transmitted into the body, they interact with the tissues and organs present in the body and are reflected back to the probe. Probe 110 receives these reflected signals and processor 120 uses mathematical algorithms to calculate the distance, composition, and density of the tissues and organs based on the time delay and intensity of the reflected signals.

The mathematical algorithms used by the processor 120 may include but are not limited to: time delay estimation techniques such as cross-correlation or the chirp-z transform, and intensity-based techniques such as pulse-echo or continuous-wave imaging. These techniques allow the processor 120 to generate a 3D map of the human anatomy with high accuracy and resolution.

Referring in particular to FIG. 2, once the 3D map is generated, processor 120 uses machine learning (ML) algorithms to analyze the map for any abnormalities or diseases present in the body. These algorithms may include, but are not limited to, supervised learning techniques that are trained on a dataset of healthy and diseased individuals, and unsupervised learning techniques that can identify patterns and anomalies in the 3D map without the need for a training dataset. The ML algorithms also may use various feature extraction techniques, such as wavelet transformation or principal component analysis, to extract relevant features from the 3D map and improve the accuracy of the disease detection.

Summarizing to this point, the present disclosure provides a system and method for 3D mapping of the human or animal anatomy for disease detection using high frequency technology is based on the principles of wave propagation and machine learning and employs various mathematical algorithms to accurately generate and analyze the 3D map of the human or animal anatomy for the detection of any abnormalities or diseases.

Various techniques can be used for analyzing the reflected signals to generate the 3D map of the target structure. These include:

### Time Delay Estimation Techniques

Cross-correlation: R(t)=∫s1(t)*s2(t−δ) dt where R(t) is the cross-correlation function, s1(t) and s2(t) are the signals transmitted and received by the probe, and δ is the time delay between the signals. The time delay δ can be calculated by finding the peak of the cross-correlation function R(t).

Chirp-z transformation: X(k)=Σx(n)*W_N\hat{ }(nk) where X(k) is the chirp-z transform of the signal x(n), W_N is the N-th root of unity, and n and k are integers. The time delay δ can be calculated by finding the peak of the chirp-z transform X(k) at the corresponding frequency k.

### Intensity-Based Techniques

Pulse-echo imaging: I(r, t)=∫(t−2r/c)*P(t) dt where I(r, t) is the intensity of the reflected signal at distance r and time t, S(t) is the echo signal received by the probe, P(t) is the transmitted pulse, and c is the speed of sound in the medium. The distance r can be calculated by finding the peak of the intensity I(r, t) at the corresponding time t.

Continuous-wave imaging: I(r)=S(r)*P(r) where I(r) is the intensity of the reflected signal at distance r, S(r) is the echo signal received by the probe, and P(r) is the transmitted continuous wave. The distance r can be calculated by finding the peak of the intensity I(r).

### Machine Learning Algorithms

Supervised learning: f(x)=w\hat{ }T*x+b where f(x) is the predicted output, x is the input feature vector, w is the weight vector, and b is the bias term. The weight vector w and bias term b are learned from a training dataset of healthy and diseased individuals by minimizing the loss function L(w, b).

Unsupervised learning: J=Σ(x−μ)\hat{ }2 where J is the objective function, x is the input data, and μ is the centroid of the data. The centroid μ can be found by minimizing the objective function J using an optimization algorithm, such as the k-means algorithm.

### Feature Extraction Techniques

Wavelet transformation: W(a, b)=∫f(t)*ψ*((t−b)/a) dt where W(a, b) is the wavelet coefficient, f(t) is the input signal, ψ(t) is the wavelet function, and a and b are scaling and translation parameters. The wavelet coefficient W(a, b) can be calculated for different values of a and b to extract features from the signal f(t).

Principal component analysis: z=W\hat{ }T*x where z is the transformed feature vector, x is the input feature vector, and W is the transformation matrix. The transformation matrix W is calculated from the input data x by finding the eigenvectors of the covariance matrix of x.

### Optimization Algorithms

K-means algorithm: μ_k=(1/n_k)*Σx_i where μ_k is the centroid of the k-th cluster, n_k is the number of points in the k-th cluster, and x_i is the i-th point in the dataset. The centroids μ_k are updated iteratively by assigning each point x_i to the closest centroid and recalculating the centroids based on the assigned points.

### Data Analysis Techniques

Statistical analysis: μ=(1/n)*Σx_i where μ is the mean of the data, n is the number of data points, and x_i is the i-th data point. The mean μ can be used to describe the central tendency of the data and to identify any outliers.

Data visualization: y=a*x+b where y is the dependent variable, x is the independent variable, and a and b are constants. A linear regression model such as this one can be used to visualize the relationship between two variables and to make predictions about the dependent variable y based on the independent variable x.

### Signal Processing Techniques

Filtering: y(n)=Σh(k)*x(n−k) where y(n) is the filtered signal, x(n) is the input signal, and h(k) is the impulse response of the filter. The filtered signal y(n) can be calculated by convolving the input signal x(n) with the impulse response h(k).

Noise reduction: y(n)=x(n)−μ where y(n) is the noise-reduced signal, x(n) is the input signal, and μ is the mean of the input signal. The noise-reduced signal y(n) can be obtained by subtracting the mean μ from the input signal x(

In the context of artificial intelligence:

### Image Enhancement

Histogram equalization: H(r_k)=(L−1)*Σp(r_i) for i=0 to k where H(r_k) is the equalized histogram value at intensity level r_k, p(r_i) is the probability of intensity level r_i in the input image, and L is the number of intensity levels. The equalized histogram H(r_k) can be calculated by accumulating the probabilities p(r_i) of the input image and scaling the result to the range [0, L−1].

Image sharpening: g(x, y)=f(x, y)+α*∇\hat{ }2 f(x, y) where g(x, y) is the sharpened image, f(x, y) is the input image, ∇\hat{ }2 f(x, y) is the Laplacian of the image, and α is a constant. The sharpened image g(x, y) can be obtained by adding the Laplacian of the input image to the input image, with a scaling factor α.

### Image Restoration

Deblurring: g(x, y)=∫f(x, y)*h(u, v) du dv where g(x, y) is the restored image, f(x, y) is the degraded image, and h(u, v) is the point spread function. The restored image g(x, y) can be obtained by convolving the degraded image f(x, y) with the point spread function h(u, v).

Denoising: g(x, y)=argmin Σ(f(x, y)−g(x, y))\hat{ }2+λΣ|∇g(x, y)|\hat{ }2 where g(x, y) is the denoised image, f(x, y) is the noisy image, and λ is a constant. The denoised image g(x, y) can be obtained by minimizing the objective function, which consists of a data fidelity term and a regularization term. The data fidelity term measures the difference between the noisy image f(x, y) and the denoised image g(x, y), while the regularization term promotes smoothness in the denoised image.

Mathematical equations that may be used by the system for real-time visible image topographical reconstruction using artificial intelligence:

### Depth Estimation

Stereo matching: d(x, y)=argmin Σ|I_l(x, y)−I_r(x−d(x, y), y)| where d(x, y) is the estimated depth at pixel (x, y), I_l(x, y) and I_r(x, y) are the intensities of the left and right images at pixel (x, y), and the sum is taken over a local neighborhood around (x, y). The depth d(x, y) can be estimated by minimizing the difference between the intensities of the left and right images for a given pixel (x, y) and its neighbors.

Machine learning-based depth estimation: d(x, y)=f(I(x, y)) where d(x, y) is the estimated depth at pixel (x, y), I(x, y) is the intensity of the input image at pixel (x, y), and f( ) is a machine learning model trained to predict the depth from the intensity. The depth d(x, y) can be estimated using a machine learning model that has been trained on a dataset of images and corresponding depth maps.

### 3D Reconstruction

Structure from motion: P=Σa_i*X_i where P is the projection matrix, a_i is the i-th element of the camera parameter vector, and X_i is the i-th 3D point. The projection matrix P can be obtained by minimizing the projection error between the 3D points X_i and their projections in the images, using an optimization algorithm such as the Levenberg-Marquardt algorithm.

Multi-view stereo: X=argmin Σ|P_i*X−p_i|\hat{ }2 where X is the 3D point cloud, P_i is the projection matrix for the i-th view, and p_i is the 2D point correspondences in the i-th view. The 3D point cloud X can be obtained by minimizing the projection error between the 3D points and their projections in the images, using an optimization algorithm such as the Levenberg-Marquardt algorithm.

Deep learning-based depth estimation: d(x, y)=f_θ(I(x, y)) where d(x, y) is the estimated depth at pixel (x, y), I(x, y) is the intensity of the input image at pixel (x, y), and f_θ( ) is a deep learning model with parameters θ. The depth d(x, y) can be estimated using a deep learning model that has been trained on a large dataset of images and corresponding depth maps. The model can be a convolutional neural network (CNN) or a recurrent neural network (RNN), depending on the specific needs and requirements of the system.

Graph-based 3D reconstruction: X=argmin Σ(p_i−P_i*X)\hat{ }T*W_i*(p_i−P_i*X) where X is the 3D point cloud, p_i is the 2D point correspondences in the i-th view, P_i is the projection matrix for the i-th view, and W_i is a weight matrix for the i-th view. The 3D point cloud X can be obtained by minimizing the reprojection error between the 3D points and their projections in the images, using a graph-based optimization algorithm such as the bundle adjustment algorithm. The weight matrix W_i can be used to balance the contribution of the different views to the optimization process.

Learning-based 3D reconstruction: X=argmin Σ(p_i−P_i *X)\hat{ }T*W_i*(p_i−P_i*X)+λΣ|X−X_0|\hat{ }2 where X is the 3D point cloud, p_i is the 2D point correspondences in the i-th view, P_i is the projection matrix for the i-th view, W_i is a weight matrix for the i-th view, λ is a constant, and X_0 is the initial estimate of the 3D point cloud. The 3D point cloud X can be obtained by minimizing the reprojection error between the 3D points and their projections in the images, while also regularizing the solution towards the initial estimate X_0. This approach can be used to incorporate prior knowledge or constraints into the reconstruction process.

Generative modeling-based 3D reconstruction: p(X)=Πp(x_i|x_{i−1}, . . . , x_1) where p(X) is the probability of the 3D point cloud X, and p(x_i|x_{i−1}, . . . , x_1) is the conditional probability of the i-th point x_i given the previous points x_{i−1}, . . . , x_1. The 3D point cloud X can be generated by sampling from the probability distribution p(X) using a Monte Carlo method such as the Markov chain Monte Carlo (MCMC) algorithm. This approach can be used to generate multiple plausible 3D reconstructions of the scene and to incorporate uncertainty into the reconstruction process.

Sparse coding-based 3D reconstruction: X=argmin Σ|x_i−D*a_i|\hat{ }2+λΣ|a_i| where X is the 3D point cloud, x_i is the i-th input image, D is the dictionary matrix, a_i is the coefficient vector for the i-th image, and λ is a constant. The 3D point cloud X can be obtained by minimizing the reconstruction error between the input images x_i and their reconstructions D*a_i, using a sparsity-promoting regularization term λΣ|a_i|. This approach can be used to represent the 3D point cloud X as a linear combination of a dictionary of 3D bases and to reduce the redundancy in the representation.

Dense correspondence-based 3D reconstruction: X=argmin Σ|x_i−X_i|\hat{ }2+λΣ|X_i−X_{i−1}|\hat{ }2 where X is the 3D point cloud, x_i is the i-th input image, and λ is a constant. The 3D point cloud X can be obtained by minimizing the photometric error between the input images x_i and their projections X_i in the 3D space, using a smoothness-promoting regularization term λΣ|X_i−X_{i−1}|\hat{ }2. This approach can be used to find dense correspondences between the input images and to reconstruct a smooth and consistent 3D point cloud.

While the foregoing disclosure has been described primarily in connection with 3D mapping of the trachea and lungs for diagnosing and treatment of various lung diseases, the disclosure is not limited. Rather the device, system and method also may be used in connection with 3D mapping of the throat and also the thyroid for diagnosing and treatment of throat and thyroid cancer, goiter and nodules. The device system and method also may be used for real-time 3D anatomy mapping of other internal body structures including by way of example, but not limited to the heart, liver, pancreas, kidneys, bladder, stomach, intestine, brain and arteries.

Various other changes are possible. For example, we can use ultrafast imaging techniques such as ultrafast laser scanning or swept-source optical coherence tomography (OCT) to capture high-resolution 3D images of a human's or animal's anatomy at very high frame rates. This would allow the system to track fast-moving or dynamic structures in real time and to provide a detailed, up-to-date model of the anatomy.

Another approach would involve using machine learning algorithms to analyze and interpret the high-frequency images in real time. This could include techniques such as deep learning, which can recognize patterns and features in the images and make predictions about the anatomy or disease status.

In yet another modification we could use a combination of different imaging modalities, such as ultrasound, magnetic resonance imaging (MRI), and computed tomography (CT), to provide a more comprehensive view of the anatomy. By combining the strengths of different modalities, the system could provide more accurate and detailed 3D maps of the anatomy.

1. A device for non-invasive 3D mapping of target internal body structure of a human or animal, comprising:
a probe configured for emitting high frequency waves;
a receiver for receiving the reflected waves;
a processing unit for calculating the distance between the probe and receiver based on the time delay of the reflected waves; and
a display for presenting a 3D map of the target structures based on the calculated distances.

2. The device of claim 1, wherein the high frequency waves are in the range of 100 MHz to 10 GHz.

3. The device of claim 1, further comprising a user interface for adjusting the frequency and intensity of the high frequency waves.

4. The device of claim 1, wherein the target body structures comprise the trachea or lungs.

5. The device of claim 4, further comprising a positioning system for aligning the device with the trachea and lungs.

6. The device of claim 1, wherein the target body structures are selected from the group consisting of the heart, liver, pancreas, kidneys, bladder, stomach, intestines, brain and arteries.

7. The device of claim 1, wherein the target body structures comprise the throat or thyroid.

8. A method for non-invasive 3D mapping of the target internal body structures of a human or animal using a device as claimed in claim 1, comprising the steps of:
placing the probe in contact with the skin of the human or animal;
emitting high frequency waves from the probe in the direction of the target body structure;
receiving the reflected waves from the target body structure with the receiver;
calculating the distance between the probe and receiver based on the time delay of the reflected waves; and
coating a 3D map of the target body structure on the display based on the calculated distances.

9. The method of claim 8, wherein the distance between the probe and the receiver is calculated using one or more of the following time delay estimation techniques: Cross-correlation; Chirp-Z transformation; Pulse-echo imaging; and Continuous-wave imaging.

10. The method of claim 8, wherein the 3D mapping is created using one or more image enhancement techniques, image sharpening techniques, image restoration techniques, and deposing techniques on a processing unit.

11. The method of claim 8, wherein the 3D map is created using one or more of the following techniques: Structure from motion; Multi-view stereo; Deep learning-based depth stimulation; Graph-based 3D reconstruction; Learning-based 3D reconstruction; Generative modeling-based 3D reconstruction; Sparce coding-based 3D reconstruction; and Dense correspondence-based 3D reconstruction.
